(19) United States
(12) Patent Application Publication
(10) Pub. No.: US 2025/0275693 A1
(43) Pub. Date: Sep. 4, 2025

(54) STRETCHABLE SENSOR ASSEMBLY

(71) Applicant: GE Precision Healthcare LLC, Waukesha, WI (US)

(72) Inventors: Matti Kosonen, Jarvenpaa (FI); Aaro Pallasmaa, Helsinki (FI)

(21) Appl. No.: 18/592,165

(22) Filed: Feb. 29, 2024

(51) Int. Cl. A61B 5/1455; A61B 5/00

(52) U.S. Cl. CPC A61B 5/14552; A61B 5/6826; A61B 2562/166

(57) ABSTRACT

Systems are provided for a device comprising an emitter, a detector, a power/signal cable directly connected to one of the detector or the emitter, and a wave-shaped flexible printed circuit board (PCB) disposed between the emitter and the detector, wherein the wave-shaped flexible PCB includes a first plurality of conductive traces. The device may further comprise an elastic cover which makes the device a stretchable sensor assembly.

### TECHNICAL FIELD

Embodiments of the subject matter disclosed herein relate to a stretchable assembly for adjusting a distance between two components of the stretchable sensor assembly.

### BACKGROUND

In some examples a finger pulse oximeter sensor may be used for measuring saturation of oxygen in blood of a patient (SpO2). The finger sensor may include a light emitting diode (LED) emitter adapted to be positioned on a first side of the finger and a detector adapted to be positioned on a second side of the finger, opposite the first side. For accurate oxygen saturation measurements, the emitter and detector are aligned such that light from the emitter passes through the finger and interacts with the detector. When the finger is a larger or smaller diameter than expected, the emitter and detector may become misaligned, leading to inaccurate determination of oxygen saturation in the blood.

### BRIEF DESCRIPTION

In one embodiment, a device, comprising: an emitter; a detector; a power/signal cable directly connected to one of the detector or the emitter; and a wave-shaped flexible printed circuit board (PCB) disposed between the emitter and the detector, wherein the wave-shaped flexible PCB includes a first plurality of conductive traces.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, herein below:

FIG. 1 shows an example from the prior art of a non-stretchable sensor assembly on a large subject.

FIG. 2 shows an example from the prior art of the non-stretchable sensor assembly on a small subject.

FIG. 3 shows an example of a stretchable sensor assembly fitted to a large subject.

FIG. 4 shows an example of the stretchable sensor assembly fitted to a small subject.

FIG. 5 shows a perspective view of hardware of a stretchable sensor assembly in a compact state.

FIG. 6 shows the perspective views of hardware of the stretchable sensor assembly in a stretched state.

FIG. 7 shows a perspective view of a stretchable sensor assembly in a compact state.

FIG. 8 shows a side view of the stretchable sensor assembly in the compact state.

FIG. 9 shows a side view of the stretchable sensor assembly in a stretched state.

FIG. 10 shows a first view of the stretchable sensor assembly positioned on a finger.

FIG. 11 shows a second view of the stretchable sensor assembly positioned on the finger.

FIG. 12 shows a block diagram illustrating an example pulse oximetry system.

### DETAILED DESCRIPTION

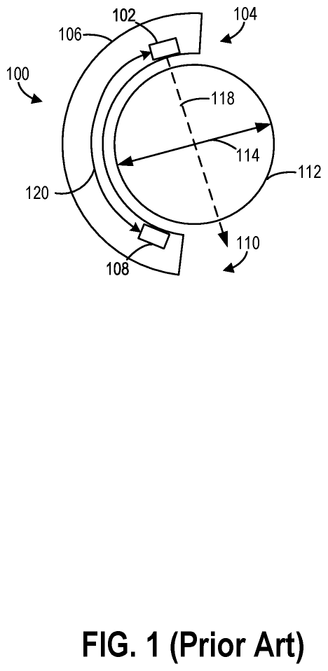

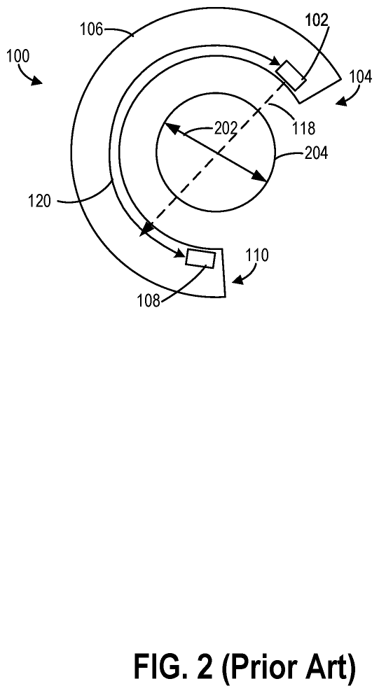

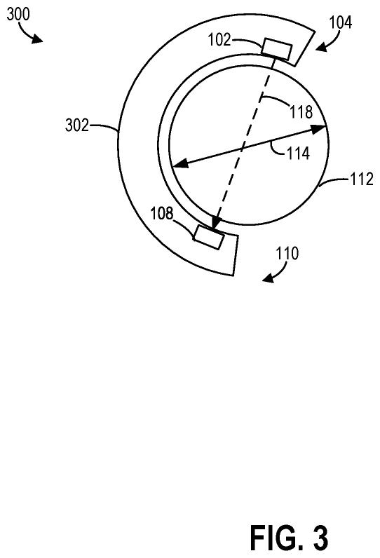

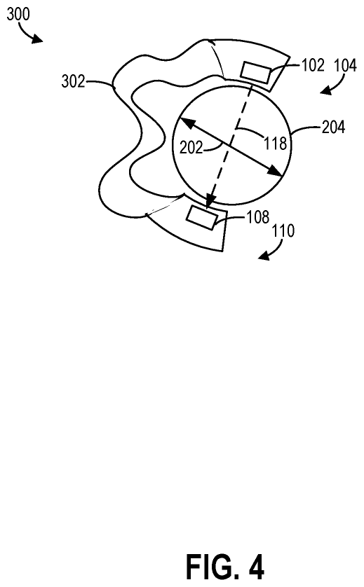

The following description relates to a stretchable sensor assembly. In an exemplary embodiment, the stretchable sensor is a finger pulse oximetry sensor, however other types of sensors are also considered. A sensor, such as a finger pulse oximetry sensor, may include two components which demand a relative alignment in order to sense with reduced noise and power consumption, and thus produce an accurate reading. In examples of the prior art, shown in FIGS. 1-2, the components of a non-stretchable sensor assembly may become unaligned when fitted to a target, such as finger, which is either larger (see FIG. 1) or smaller (see FIG. 2) than the intended target size. The issues shown in FIGS. 1-2 may be at least partially addressed by a stretchable sensor assembly. The stretchable sensor assembly may be fitted in a stretched state when the target is large, as shown in FIG. 3 or may be fitted in a compact state when the target is small, as shown in FIG. 4.

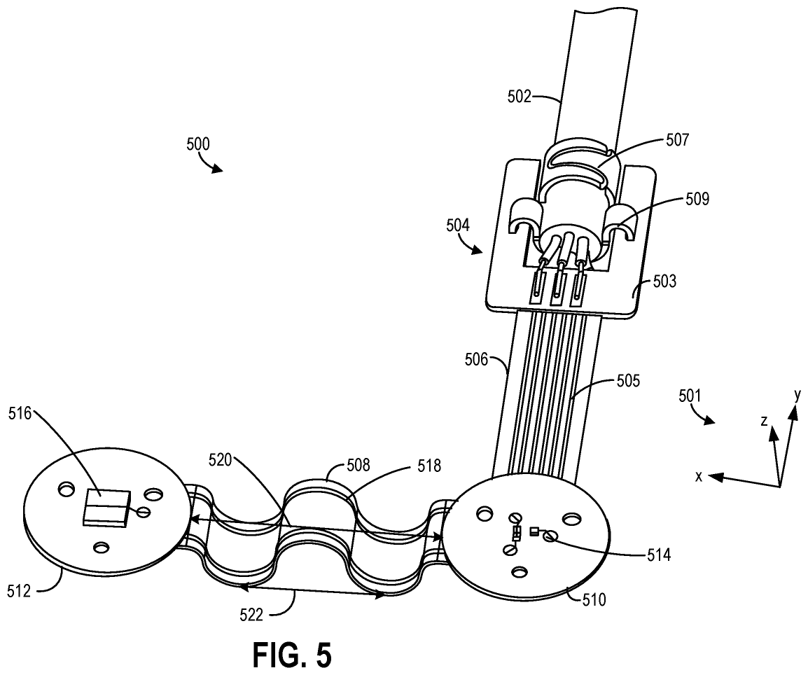

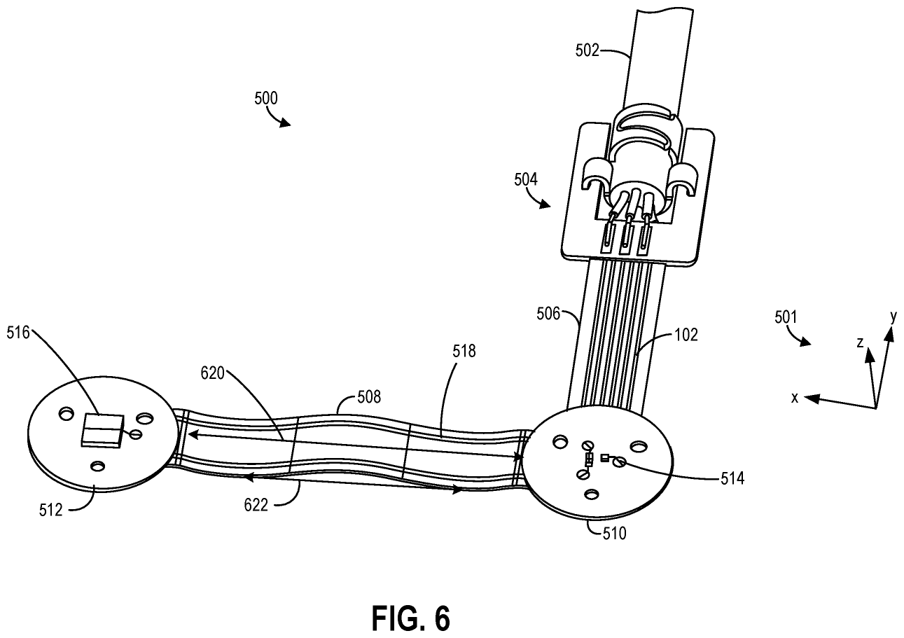

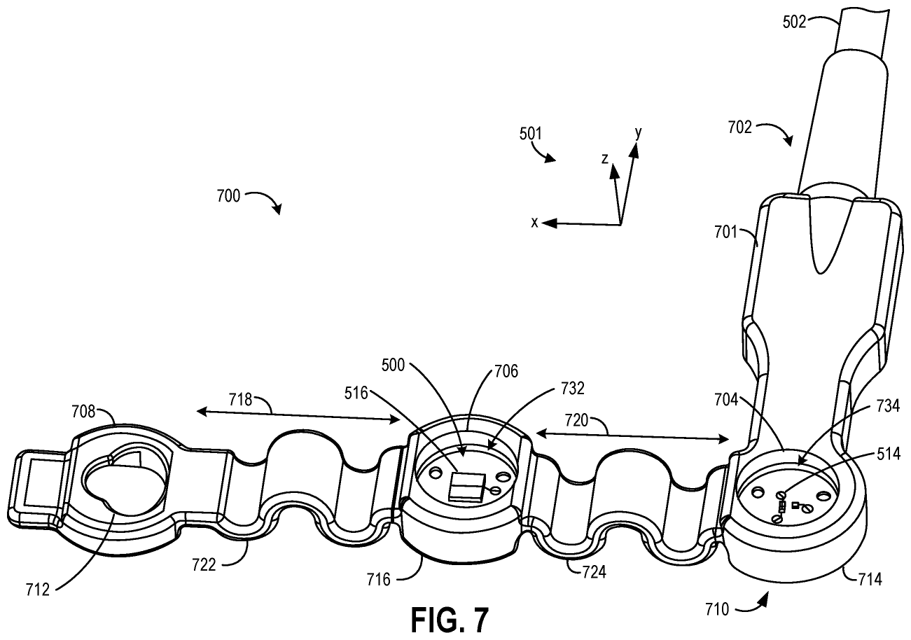

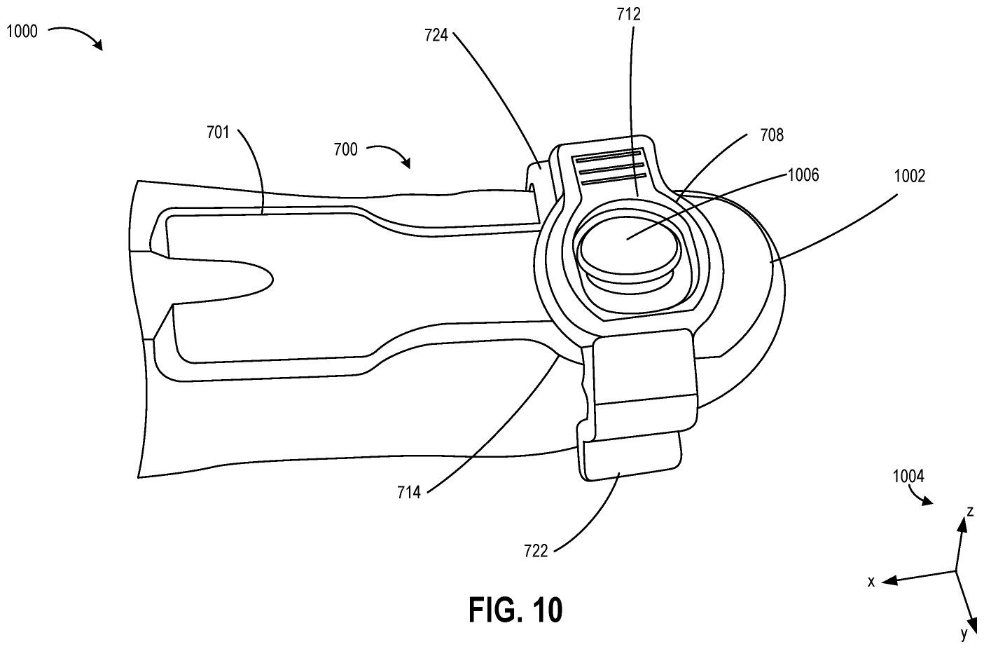

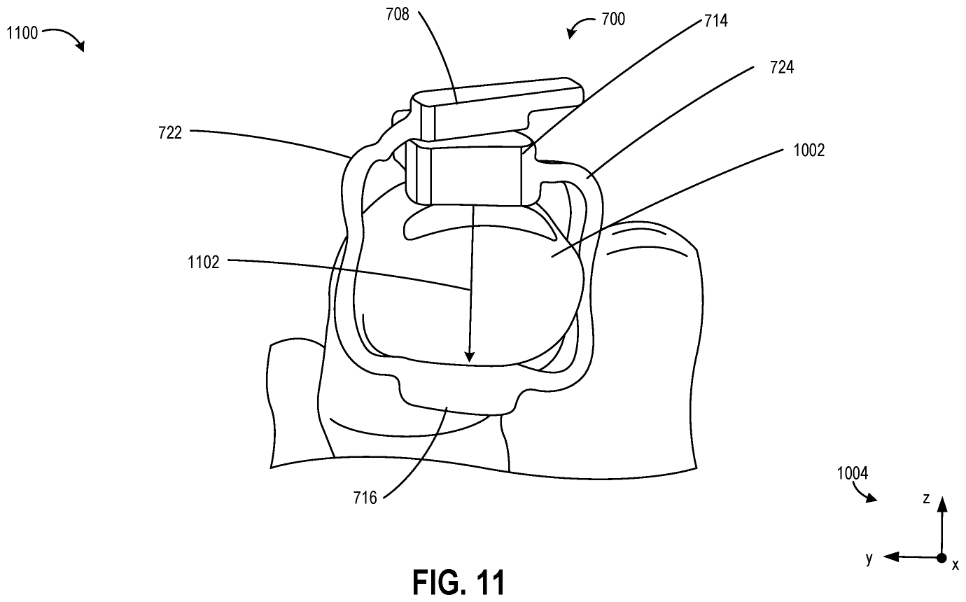

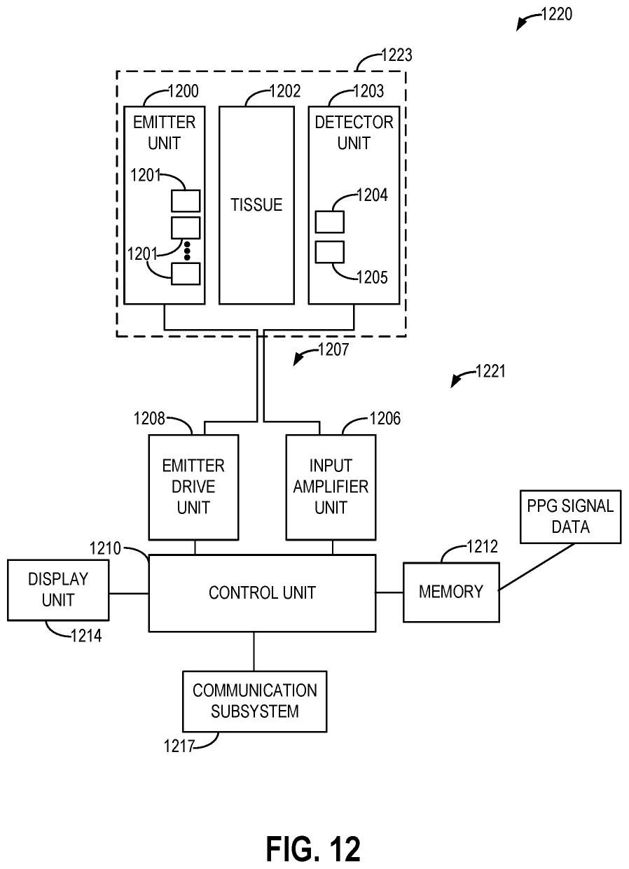

In the exemplary embodiment where the stretchable sensor is a finger pulse oximetry sensor such as shown as a schematic representation in FIG. 12, the sensor may include hardware as shown in FIG. 5-6. The hardware may include components affixed to rigid-flexible printed circuit board (PCB) and/or flexible PCB with stiffener positioned in some portions. A portion of the sensor assembly electrically coupling the two components may be a flexible PCB and may be adapted to be stretchable. In this way, the two components are both electrically coupled via each other to a power/signal cable, and separate couplers are not demanded for each of the two components such that a single coupler may electrically couple the two components or a plurality of components the same configuration as the two components. By not demanding separate couplers, the sensor assembly is compact and is not prone to snagging on other hospital equipment. The hardware may be further covered by an elastic sleeve to form the stretchable sensor assembly shown in FIGS. 7-9. The stretchable sensor assembly may be positioned around a finger to sense saturation of oxygen in the blood as shown in FIGS. 10-11.

Turning now to FIG. 1, it shows a non-stretchable sensor assembly 100 of the prior art. The non-stretchable sensor assembly includes a first component 102 affixed to a first end 104 of a body 106. A second component 108 may be affixed towards a second end 110 of the non-stretchable sensor assembly 100. The body 106 may be non-stretchable, holding the first component 102 at a fixed distance 120 (along a length of the body 106) from the second component 108. The non-stretchable sensor assembly may be positioned on a large target 112. As one example, the large target 112 may be circular and may have a diameter 114.

In an example where the non-stretchable sensor assembly 100 is a finger pulse oximeter, the first component 102 may be an LED array adapted to emit collimated light 118 and the large target 112 may be a finger. The collimated light 118 may travel through the finger. When positioned on a medium sized finger with a diameter smaller than diameter 114, collimated light 118 may pass through the finger and hit the second component 108 which may be a photodetector. In this way, light may pass through the finger, interact with hemoglobin in the blood and be detected by the detector to determine SpO2. However, when diameter 114 is large as is shown in FIG. 1, the body 106 may experience a shape change to fit over the large diameter, including deflection in a radial direction from the large target 112, causing the detector (e.g., second component 108) to not be positioned directly in the path of collimated light 118. Consequently, an accuracy of the determined SpO2 may be decreased.

The first component and second component may also be unaligned if the small target 204 has a small diameter 202 as shown in FIG. 2. The small diameter 202 may be smaller than a medium sized target (e.g., finger). Shape change to the body 106 to fit small diameter 202 may also cause collimated light 118 to be misaligned with the detector (e.g., second component 108). For example, when misaligned, collimated light 118 or another beam projected from the first component 102 may not be centered along an axis to touch an approximate detection point of the second component 108 or be outside a cone of detection extending radially from the detection point of the second component 108. A non-stretchable sensor array may be provided in more than one size, to account for different sized targets. However, keeping a stock of different sized sensors increases a cost and complexity of using the sensor in a setting, such as a hospital setting, where patients of many different sizes are seen.

A stretchable sensor assembly, such as stretchable sensor assembly 300 shown in FIGS. 3 and 4 may be used as a one size fits all sensor assembly. The stretchable sensor assembly 300 may be used more accurately than the non-stretchable sensor assembly of FIGS. 1 and 2 with both a small target and a large target. FIG. 3 shows the large target 112 of FIG. 1 fitted with a stretchable sensor assembly 300 in a stretched configuration. Stretchable sensor assembly 300 may include first component 102 and second component 108. Each of first component 102 and second component 108 of stretchable sensor assembly 300 may be affixed to opposite ends of a stretchable band 302. As shown in FIG. 3, stretchable band 302 may be in a fully stretched state, thereby extending a distance between first component 102 and second component 108. In this way, first component 102 and second component 108 may be aligned. In the example where stretchable sensor assembly 300 is a finger pulse oximetry sensor, collimated light 118 from the emitter may reach the detector and SpO2 may be determined more accurately than when collimated light 118 is misaligned from the detector.

Stretchable band 302 may be shown in a compact state in FIG. 4, in which stretchable band 302 is collapsed compared to the stretched state shown in FIG. 3. The compact state may include stretchable band 302 being collapsed or folded into an accordion shape or a wave shape, thereby decreasing the distance between first component 102 and second component 108. Thus, first component 102 may be aligned with second component 108 when fitted to small target 204. For example, when the stretchable band 302 stretched or collapsed/folded, the first component 102 and second component 108 may remain approximately centered on the same axis as before the stretchable band 302 is stretched or folded. The collimated light 118 may be projected approximately along or in parallel with the axis.

In contrast with the non-stretchable sensor assembly 100 of prior art shown in FIGS. 1 and 2, the stretchable sensor assembly 300 may fit both large target 112 and small target 204 such that first component 102 and second component 108 may be aligned. In this way, the stretchable sensor assembly 300 may fit a range of target sizes (e.g., a range between and including sizes of small target 204 and large target 112) and prevent misalignment of first component 102 and second component 108. In some examples, stretchable band 302 may be elastic, such that the compact state may be a resting state of stretchable band 302. The stretchable band 302 may return to the compact state when not under tension, and the stretched state may be reached by applying tension to stretchable band 302. The stretchable band 302 may have a plurality of stretched states, where the increasing tension between a first stretched state and a second stretched state may increase the distance the stretchable band 302 is stretched. Thus, stretchable band 302 may experience shape change from the compact state, or resting state, to accommodate larger target sizes and promote alignment of first component 102 and second component 108. In the example where stretchable sensor assembly 300 is a finger pulse oximeter, first component 102 may be a light emitter that emits light which may be received by second component 108 due to the alignment thereof after traveling through the target, for a range of target sizes (e.g., a range of finger sizes).

As described above, in some embodiments, a stretchable sensor assembly may be a finger pulse oximetry sensor. FIG. 12 shows a block diagram of one embodiment of a pulse oximetry system 1220. Light transmitted from an emitter unit 1200 passes into patient tissue 1202. The emitter unit includes multiple light sources 1201, such as light-emitting diodes (LEDs), with each light source having a dedicated wavelength. Each wavelength forms one measurement channel on which photoplethysmography (PPG) waveform data are acquired.

The light transmitted through the patient tissue 1202 is received by a detector unit 1203, which comprises two photodetectors 1204 and 1205 in this example. For example, photodetector 1204 may be a silicon photodiode, and photodetector 1205 may be a second silicon photodiode with different spectral characteristics or an indium gallium arsenide (InGaAs) photodiode. The emitter and detector units form a probe subunit 1223 of the pulse oximetry system 1220.

The probe subunit 1223 may be coupled (e.g., electrically and/or communicatively) to a drive and processing subunit 1221 via a cable 1207 and one or more connectors. For example, a connector may be present on an end of cable 1207 to connect cable 1207 and probe subunit 1223 to drive and processing subunit 1221. In this way, probe subunit 1223 may be removably coupled to drive and processing subunit 1221, such that probe subunit 1223 may be communicatively coupled and decoupled from the drive and processing subunit 1221. In other examples, probe subunit 1223 and drive and processing subunit 1221 may be integrated into the same housing.

Drive and processing subunit 1221 may include an input amplifier unit 1206 and an emitter drive unit 1208. The photodetectors convert the optical signals received into electrical pulse trains and feed them to an input amplifier unit 1206. The amplified measurement channel signals are further supplied to a control unit 1210, which executes instructions stored in memory 1212 to convert the signals into digitized format for each wavelength channel.

The control unit 1210 further controls emitter drive unit 1208 to alternately activate the light sources. To activate the light sources, the emitter drive unit 1208 may include a voltage source, such as a battery, described in more detail below. As mentioned above, each light source is typically illuminated several hundred times per second. With each light source being illuminated at such a high rate compared to the pulse rate of the patient, the control unit 1210 obtains a high number of samples at each wavelength for each cardiac cycle of the patient. The value of these samples varies according to the cardiac cycle of the patient, where the variation may be caused by the arterial blood.

The digitized PPG signal data at each wavelength may be stored in memory 1212 of the control unit 1210 before being processed further according to non-transitory instructions (e.g., algorithms) executable by the control unit 1210 to obtain physiological parameters. Memory 1212 may comprise a suitable data storage medium, for example, a permanent storage medium, removable storage medium, and the like. Additionally, memory 1212 may be a non-transitory storage medium. In some examples, the pulse oximetry system 1220 may include a communication subsystem 1217 operatively coupled to one or more remote computing devices, such as hospital workstations, smartphones, and the like. The communication subsystem 1217 may enable the output from the detector units (e.g., the digitized PPG signal data) to be sent to the one or more remote computing devices for further processing, and/or the communication subsystem may enable the output from the algorithms discussed below (e.g., determined physiological parameters) to be sent to the remote computing devices. The communication subsystem 1217 may include wired and/or wireless communication devices compatible with one or more different communication protocols. As non-limiting examples, the communication subsystem 1217 may be configured for communication via a wireless telephone network, a local-area or wide-area network, and/or the Internet.

Algorithms may utilize the same digitized signal data and/or results derived from the algorithms and stored in the memory 1212, for example. For example, to determine oxygen saturation and pulse transit time (PTT), the control unit 1210 may be adapted to execute an SpO2 algorithm and a PTT algorithm, respectively. For this example, the SpO2 algorithm and a PTT algorithm may be stored in the memory 1212 of the control unit 1210. Additional algorithms, such as a blood pressure algorithm, a hypovolemia algorithm, and a respiration rate algorithm, may also be stored in memory 1212 for determining blood pressure, an indication of hypovolemia, and respiration rate, respectively. The obtained physiological parameters and waveforms may be shown on a screen of a display unit 1214. Further, in some examples, the control unit, memory, and/or other subsystems may be located remotely from the rest of the sensor on a separate device, and the signal data from the detector units may be sent to the separate device for processing.

The input amplifier unit 1206, the control unit 1210 and memory 1212, the emitter drive unit 1208, probe subunit 1223, and/or additional components (the display unit, for example) may collectively form a sensor. As used herein, the term “probe” may refer to the probe and the attachment parts that attach the optical components of the probe to the tissue site. The term pulse oximeter or pulse oximetry sensor may refer to a unit comprising a probe, an analog front end, and a signal processing unit that calculates SpO2 and other blood characteristics. In a multi-parameter body area network system, the system typically represents a set of multiple sensors, e.g., the different physiological parameter measurements. Therefore, the whole measurement system may comprise several sensors, and the sensors may communicate to a common hub in which the parameters' information is integrated.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

An example of pulse oximetry hardware 500 is shown in FIGS. 5-6. Pulse oximetry hardware 500 may include the probe subunit 1223 and the connect cable 1207 of FIG. 12. Additionally or alternatively, the pulse oximetry hardware may be incorporated into a stretchable pulse oximetry sensor assembly, such as stretchable sensor assembly 700 as shown in FIGS. 7-11. Reference axes 501 are provided for comparison between the views shown in FIGS. 5-9, including an x-axis, a y-axis, and a z-axis.

Pulse oximetry hardware 500 may include a cable 502 (e.g., the connect cable 1207 of FIG. 12). Cable 502 may couple pulse oximetry hardware 500 to a power source (not shown), supplying power to components of pulse oximetry hardware 500. Additionally or alternatively, cable 502 may communicatively couple pulse oximetry hardware 500 to a controller (e.g., drive and processing subunit 1221 of FIG. 12) such that the pulse oximetry hardware may send and receive signals from the controller. The controller may include instructions to emit light and interpret signals from a detector to provide a user with a quantitative SpO2 of a patient wearing pulse oximetry hardware 500, for example around the finger of the patient or another example of the large target 112 or the small target 204 of FIGS. 1-4. Cable 502 may be a power/signal cable which includes a plurality of wires 504. In other examples, cable 502 may be a battery and transmission unit that allows for wireless operation of pulse oximetry hardware 500. The plurality of wires 504 may be mounted to a first rigid/stiffener portion 503, where first rigid/stiffener portion 503 may be a rigid portion of a rigid-flex-rigid PCB or a stiffener of a flex PCB with stiffeners. In this way, the rigidity of first rigid/stiffener portion 503 may prevent damage to the connections (e.g., solder joints) between the plurality of wires 504 and first rigid/stiffener portion 503. In some examples, first rigid/stiffener portion 503 may be rectangular with side extensions on which a support 507 of cable 502 is connected for physical support to the couplings between the plurality of wires 504 and first rigid/stiffener portion 503. The support 507 may wrap around cable 502, and be in face sharing contact with first rigid/stiffener portion 503. For an example, the support 507 may curve around the cable 502, such as to be positioned radially about the cable 502. The support 507 may be a sleeve or include a sleeve component. The support 507 may physically couple and/or connect to the first rigid/stiffener portion 503 via a plurality of appendages 509. The appendages 509 may be part of the support 507, such as being stamped from or molded with the support 507. The appendages 509 may be connected and/or physically coupled to the support 507, such as via joining. For an example there may be two of the appendages 509, with a first appendage opposite to and mirroring a second appendage across the support 507. In examples wherein pulse oximetry hardware 500 includes a transmission unit and a battery unit rather than wires in a power/signal cable, the transmission unit and battery unit may be coupled to the first rigid/stiffener portion 503.

First rigid/stiffener portion 503 may physically couple to a first end of a first flex portion 506, where first flex portion 506 is located opposite the plurality of wires across first rigid/stiffener portion 503, and a second end of first flex portion 506 may physically couple to a second rigid/stiffener portion 510. First flex portion 506 may be a flex portion of a flex PCB with stiffeners, or a flex portion of a rigid-flex-rigid PCB. First flex portion 506 may be ribbon shaped with a thin thickness (e.g., dimension in the z-direction), and a longer length (e.g., dimension in the y-direction, between first rigid/stiffener portion 503 and second rigid/stiffener portion 510) than width (e.g., dimension in the z-direction). Due to the flexibility of first flex portion 506, first flex portion 506 may change shape according to external forces. For example, first flex portion 506 may bend, if desired, by user manipulation for example. In another example, first flex portion 506 may be altered (e.g., bent, twisted, etc.) according to a shape of an over mold applied over first flex portion 506. Additionally, first flex portion 506 may be a single sided flexible PCB with conductive traces on one surface. Alternatively, the first flex portion 506 may be a double sided flexible PCB with conductive traces on both sides of first flex portion 506. As such, a first plurality of conductive traces 505 may extend linearly along first flex portion 506 from first rigid/stiffener portion 503 to second rigid/stiffener portion 510 (e.g., parallel to the y-axis) on one or more surfaces of first flex portion 506. The first plurality of conductive traces 505 may be conductive lines printed onto and/or etched into first flex portion 506. Each one of the first plurality of conductive traces 505 may be electrically coupled to one of the plurality of wires 504, for example via solder joints.

One or more emitters 514 (e.g., emitter unit 1200 of FIG. 12) may be positioned on a surface (e.g., in the x-y plane and facing a positive z-direction) of second rigid/stiffener portion 510. Similar to the first rigid/stiffener portion 503, second rigid/stiffener portion 510 may be a stiffener of a flex type PCB with stiffeners, or a rigid portion of a rigid-flex-rigid PCB. Emitters 514 may be mounted on second rigid/stiffener portion 510 rather than a flex portion, such as first flex portion 506, to prevent degradation of emitters 514. Second rigid/stiffener portion 510 may maintain its shape under tension, such that bending of emitters 514 is prevented, thus preventing degradation or shape change of emitters 514 and/or couplings between emitters 514 and the rigid/stiffener portion and/or electric couplings between emitters 514 and conductive traces due to shape change of the PCB. Second rigid/stiffener portion 510 may be shaped as a circular disk, as shown in FIG. 5-6, however, second rigid/stiffener portion 510 may also be other shapes including elliptical, rectangular, square, triangular, hexagonal, and the like.

In at least some examples, emitters 514 may emit light in the positive z-direction. More specifically, emitters 514 may be light-emitting diodes (LEDs). Emitters 514 may be electrically coupled to the first plurality of conductive traces 505 such that emitters 514 are electrically and/or communicatively coupled to the controller via the first plurality of conductive traces 505 and the plurality of wires 504. Thus, emitters 514 may send and/or receive power and/or signals from the controller and/or the power source via the first plurality of conductive traces 505. In this way, emitters 514 may be powered by the power source and emit light in response to signals from the controller, for example.

Second rigid/stiffener portion 510 may be connected to a first end of a second flex portion 508, and a second end of second flex portion 508 may be connected to a third rigid/stiffener portion 512. Similar to first flex portion 506, second flex portion 508 may be a flex portion of a flex PCB with stiffeners, or a flex portion of a rigid-flex-rigid PCB. Thus, a shape of second flex portion 508 may change according to conditions described above. Second flex portion 508 may be ribbon shaped, similar to first flex portion 506, with a thin thickness (e.g., in the z-direction) and a longer length (e.g., dimension in the x-direction) than width (e.g., dimension in the y-direction). When in the compact state as shown in FIG. 5, second flex portion 508 may be wave shaped, and therein be or include a wave-shaped flexible PCB. For example, a cross section of second flex portion 508 in the x-z plane may be sinusoidal, and second flex portion 508 may extend laterally in the y-direction. The flex portion 508 may include a plurality of waves when in the compact state, with a distance 522 between each of the waves of the ribbon shape of the second flex portion 508, wherein the distance 522 is not fixed such that the distance 522 may be increased when not in the compact state. The distance 522 may be parallel with the x-axis. In other examples, second flex portion 508 may be wave shaped but not sinusoidal, or have sharp peaks forming an accordion fold shape rather than a curved wave shape in the compact state. In yet other examples, second flex portion 508 may be twisted or spiraled, such as in a helical shape or a spring shape, rather than waved. In this way, the second flex portion may be shaped as a ribbon that may be adapted to change a distance 520 between second rigid/stiffener portion 510 and third rigid/stiffener portion 512. The distance 520 may be parallel with the x-axis.

The second flex portion may be a single sided flexible PCB with conductive traces on one surface, or second flex portion 508 may be a double sided flexible PCB with conductive traces on both sides of second flex portion 508. Accordingly, a second plurality of conductive traces 518 of the plurality of wires 504 may extend from second rigid/stiffener portion 510 to third rigid/stiffener portion 512. Similar to the first plurality of conductive traces 505, the second plurality of conductive traces 518 may be conductive lines printed onto and/or etched into second flex portion 508 on one or more surfaces of second flex portion 508. In at least some examples, a number of the second plurality of conductive traces 518 may be different than (e.g., greater or less than) a number of the first plurality of conductive traces 505 as shown in FIG. 5. However, the number of the first plurality of conductive traces 505 and the number of the second plurality of conductive traces 518 may be the same in other examples. In such examples, first flex portion 506 may be substantially the same as second flex portion 508. The second plurality of conductive traces 518 may run parallel to the x-axis along second flex portion 508 such that the second plurality of conductive traces 518 are wave shaped in the x-z plane in the compact state.

One or more detectors 516 (e.g., detector unit 1203 of FIG. 12) may be positioned on a surface (e.g., in the x-y plane and facing a positive z-direction) of third rigid/stiffener portion 512. Third rigid/stiffener portion 512 may be a rigid portion of a rigid-flex-rigid PCB or a stiffener of a flex PCB with stiffeners. In either case, the rigidity (e.g., resistance to deformation or shape change under tension or other forces) of third rigid/stiffener portion 512 may prevent degradation to detectors 516 and/or the physical coupling of detectors 516 to third rigid/stiffener portion 512 and/or the electric couplings between the detectors and the second plurality of conductive traces 518 due to tension on the PCB. Similar to the second rigid/stiffener portion 510, third rigid/stiffener portion 512 may be shaped as a circular disk, among other shapes as noted above according to a desired configuration. In at least some examples, the second rigid/stiffener portion 510 may be shaped approximately the same as third rigid/stiffener portion 512.

In at least some examples, detectors 516 may be photodetectors capable of detecting light, for example from LEDs, when the light is aligned with detectors 516. Additionally, detectors 516 may be able to send a corresponding electrical signal, for example to a controller which may interpret the electrical signal. Detectors 516 may be electrically coupled to the second plurality of conductive traces 518 such that detectors 516 may be powered, send signals, and/or receive signals via the second plurality of conductive traces 518.

Because of the electric couplings between the plurality of wires 504 and first plurality of conductive traces 505, between the plurality of conductive traces 505 and emitters 514, between emitters 514 and the second plurality of conductive traces 518, and between the second plurality of conductive traces 518 and detectors 516, electric signals and/or power (e.g., current) may be transmitted between emitters 514, detectors 516, and the controller via the plurality of wires 504, the first plurality of conductive traces 505, and the second plurality of conductive traces 518. In this way, detectors 516 and emitters 514 share electric couplings to the power source and controller. Thus, detectors 516 may be electrically coupled via emitters 514, such that a separate electric coupling between detectors 516 and the power source and/or the controller is not demanded. In this way, in at least some examples, detectors 516 and emitters 514 may not be electrically coupled to each other via any other electric couplers than conductive traces (e.g., first plurality of conductive traces 505 and second plurality of conductive traces 518) positioned therebetween. Thus, complexity of pulse oximetry hardware 500 may be reduced, and snagging and tangling of electric couplers (e.g., wires, power cords, etc.) may be prevented during operation of a stretchable sensor assembly in which pulse oximetry hardware 500 is incorporated.

In examples in which first rigid/stiffener portion 503, first flex portion 506, second rigid/stiffener portion 510, second flex portion 508, and third rigid/stiffener portion 512 are portions of a flex PCB with stiffeners, the stiffeners (e.g., first rigid/stiffener portion 503, second rigid/stiffener portion 510, and third rigid/stiffener portion 512) may be applied to one or both sides of the flex PCB portions (e.g., first flex portion 506 and second flex portion 508) for mechanical support, and the stiffeners may have an increased thickness than the flex PCB portions. In examples in which first rigid/stiffener portion 503, first flex portion 506, second rigid/stiffener portion 510, second flex portion 508, and third rigid/stiffener portion 512 are portions of a rigid-flex-rigid PCB, the rigid portions (e.g., first rigid/stiffener portion 503, second rigid/stiffener portion 510, and third rigid/stiffener portion 512) may serve as mechanical support and an interface for electrical couplings, and may be formed integrally with the flex portions (e.g., first flex portion 506 and second flex portion 508).

Pulse oximetry hardware 500 may be a non-limiting example of hardware incorporated in a stretchable sensor assembly, such as stretchable sensor assembly 700 of FIGS. 7-11 described below. In other examples, emitters 514 may be mounted on third rigid/stiffener portion 512 and detectors 516 may be mounted on second rigid/stiffener portion 510 such that emitters 514 are electrically coupled to cable 502 via detectors 516.

FIG. 6 shows pulse oximetry hardware 500 with second flex portion 508 in a stretched state such that a distance 620 between second rigid/stiffener portion 510 and third rigid/stiffener portion 512 is greater than the distance 520 in the compact state, shown in FIG. 5, between second rigid/stiffener portion 510 and third rigid/stiffener portion 512. Second flex portion 508 may be adjusted such that the distance 522 between waves in the compact state as shown in FIG. 5 is increased to a larger distance 622. The distance between the waves may further increase from the larger distance 622 until an unwaved shape (e.g., flat in an x-y plane) is reached for the second flex portion 508. When in an unwaved shape, the second flex portion 508 may be at a maximum distance between the second rigid/stiffener portion 510 and third rigid/stiffener portion 512, where the maximum distance is greater than the distance 620. In this way, the distance between emitters 514 and detectors 516 may be increased in the stretched state compared to the compact state shown in FIG. 5. Therefore, the distance between emitters 514 and detectors 516 may be variable. Thus, a stretchable sensor assembly (e.g., stretchable sensor assembly 700 of FIGS. 7-11) including pulse oximetry hardware 500 may be adjusted to fit a range of target sizes adequately to allow alignment of emitters 514 and detectors 516 as is shown in FIGS. 10-11 and discussed further below.

Turning to FIG. 7, it shows a stretchable sensor assembly 700. Stretchable sensor assembly 700 includes pulse oximetry hardware 500 introduced with reference to FIG. 5, and parts are labeled accordingly in FIG. 7. Pulse oximetry hardware 500 may be over molded with an elastic cover 702 such that pulse oximetry hardware 500 is encased in elastic cover 702. Elastic cover 702 may be an elastomer (e.g., natural rubber, polyurethane, polybutadiene, silicone, a mixture thereof, and the like) or other elastic material formed in the compact state, such as shown in FIG. 5. Therefore, the compact state may be a “resting” state which elastic cover 702 may return to in the absence of tension (e.g., stretching or compressing force) due to the nature of the elastic material. In this way, the ability to stretch from the compact state according to resistance of elastic cover 702 to deformation may allow for the stretchable sensor assembly to fit around a range of target sizes.

Briefly referring to FIGS. 5 and 7, elastic cover 702, which may envelop pulse oximetry hardware 500, may include a first portion 701, a second portion 714, a third portion 724, a fourth portion 716, a fifth portion 722, and a sixth portion 708. First portion 701 may be adapted to cover first rigid/stiffener portion 503 and first flex portion 506, second portion 714 may be adapted to at least partially cover second rigid/stiffener portion 510, third portion 724 may be adapted to at least partially cover second flex portion 508, and fourth portion 716 may be adapted to at least partially cover third rigid/stiffener portion 512. Fifth portion 722 may be shaped substantially the same as third portion 724 such that fifth portion 722 and third portion 724 are each a wave-shaped band, although fifth portion 722 may not cover any parts of pulse oximetry hardware 500, in at least some examples. Additionally, fifth portion 722 and third portion 724 may extend lengthwise perpendicular to one another. Sixth portion 708 may also not cover any parts of pulse oximetry hardware 500, in at least some examples.

Second portion 714 may include a first opening 734 in which a first clear cover 704 may be placed. Likewise, fourth portion 716 may include a second opening 732 in which a second clear cover 706 may be placed. The first clear cover 704 and the second clear cover 706 may be transparent so as not to interfere with transmitting light from emitters 514 to detectors 516 when the stretchable sensor assembly is positioned on a target as shown in FIGS. 10-11.

Sixth portion 708 may be used to secure stretchable sensor assembly 700 in a ring shape when positioned around a target, such as shown in FIGS. 10-11. For example, sixth portion 708 may have a complementary counterpart (not shown) located on second portion 714, such as to physically couple the second portion 714. Sixth portion 708 and the complementary counterpart may fit together in a variety of ways without departing from the scope of this disclosure. For example, the complementary counterpart may be located on a bottom 710 of second portion 714 and may extend through a hole 712 in sixth portion 708, interlocking with the hole 712 such that stretchable sensor assembly 700 forms a closed loop, or ring, around the target. In this way, elastic cover 702 may couple to itself to form a ring shape. In other examples, second portion 714 may be connected to sixth portion 708 by other means, such as snap-fit, hook and loop fasteners, and so on. By connecting second portion 714 and sixth portion 708, emitters 514 and detectors 516 may be positioned oppositely across the target. Further, the waves in third portion 724 and fifth portion 722 may align emitters 514 and detectors 516 by ensuring equidistance of spacing between emitters 514 and detectors 516 around the ring shape regardless of the target size (so long as the target size is within a target size range), as is discussed further below.

Stretchable sensor assembly 700 may be a non-limiting example of a stretchable sensor assembly, and other arrangements of elastic cover portions may be used without departing from the scope of this disclosure. For example, rather than forming an L-shape as shown in FIG. 7, a T-shape may be formed. To elaborate, a waved portion may be positioned on either side of second portion 714, rather than on either side of fourth portion 716. For example, a stretchable sensor assembly may include first portion 701, second portion 714, third portion 724, and fourth portion 716 as shown in FIG. 7, but fifth portion 722 may be positioned to the right (e.g., in a negative x-direction) of second portion 714, and sixth portion 708 may be positioned further to the right than fifth portion 722. In this example, sixth portion 708 may be adapted to couple to fourth portion 716 to form a ring shape that may be fitted to a target in a similar manner to the stretchable sensor assembly 700.

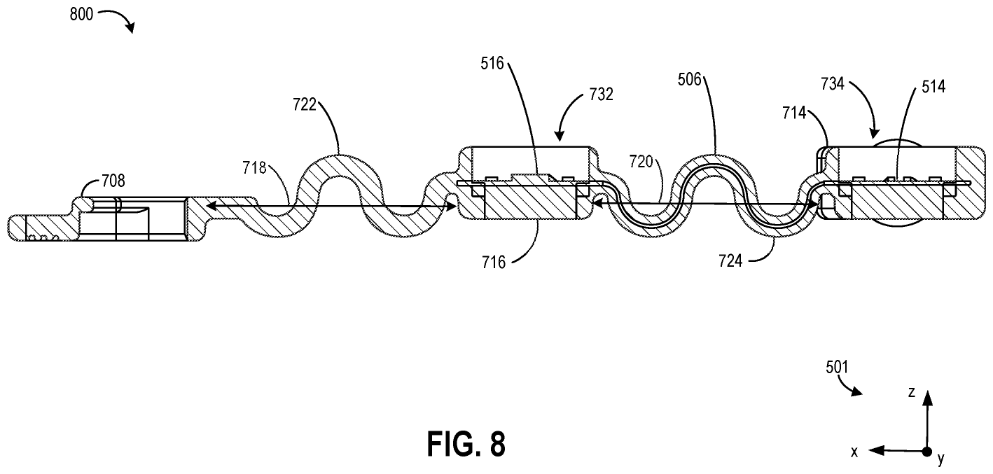

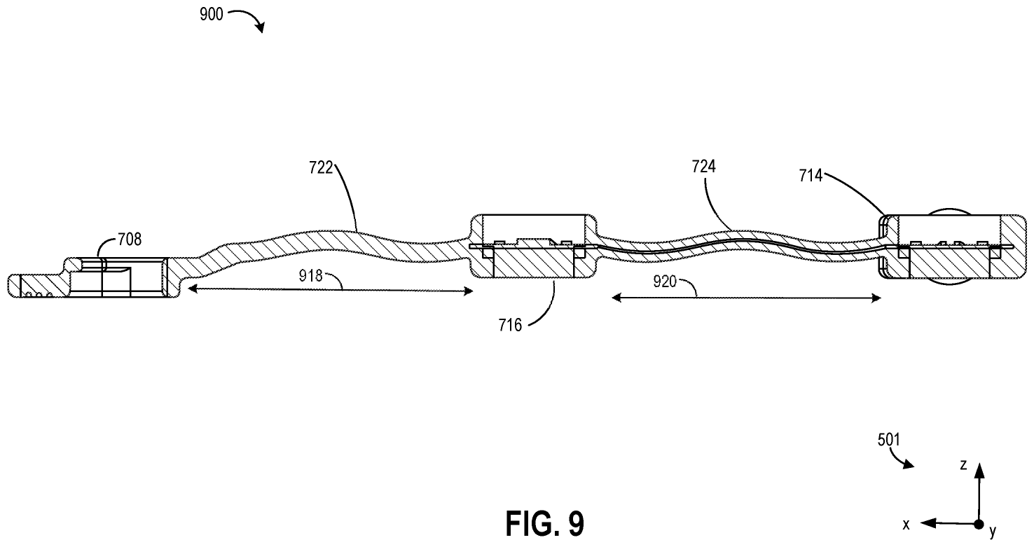

Referring to FIGS. 8 and 9, side cross section views (e.g., cross sections in an x-y plane) of stretchable sensor assembly 700 in a compact state 800 and a stretched state 900, respectively, are shown. As noted above, third portion 724 and fifth portion 722 may be approximately the same shape (e.g., wave shape) and material, thus having approximately the same resistance to deformation. Consequently, in the compact state 800, a length 720 of third portion 724 may be approximately the same as a length 718 of fifth portion 722. For example, the length 720 and the length 718 may result in a first distance between second portion 714 and fourth portion 716, and between fourth portion 716 and sixth portion 708. Likewise, in the stretched state, a length 920 of third portion 724 may be approximately the same as a length 918 of fifth portion 722. For example, the length 920 and the length 918 may result in a second distance between second portion 714 and fourth portion 716, and between fourth portion 716 and sixth portion 708. The length 920 and the length 918 may be larger than the length 720 and the length 718. In this way, when stretchable sensor assembly 700 is positioned around a subject, such as a finger, emitters 514 and detectors 516 may be aligned as is further described below.

Further, the ability to stretch third portion 724 and fifth portion 722 allows for alignment of emitters 514 and detectors 516 around a range of target sizes (e.g., finger sizes). For example, the compact state 800 may be sized appropriately for a first small target that is smaller relative to a standard target, such as a first finger of a pediatric patient, while the stretched state 900 may be able to fit a second large target that is larger relative to the standard target or the smaller target, such as a second finger of an adult patient. Another state may exist between the compact and stretched states which may fit a target of size between the sizes of the first small target and second large target, such as a finger larger than the first finger and the smaller than the second finger. Likewise, another state may exist above the compact and stretched states, such as a maximum stretched state, which may fit a target larger than the second large target, such as a finger or another feature of a subject, such as a thumb, larger than the second finger. Due to the third portion 724 and fifth portion 722 having approximately the same wave shape, emitters 514 and detectors 516 may be arranged equidistantly around the ring shape formed when positioned around, or fit to, a target. In this way, a range of target sizes (e.g., finger sizes) may be accommodated by stretchable sensor assembly 700, with emitters 514 and detectors 516 aligned for accurate measurement, for example of blood oxygen saturation.

The wave shape described herein is a non-limiting example of a stretchable shape that an elastic cover may take between emitters and detectors of a stretchable sensor assembly, such as third portion 724 and fifth portion 722 of stretchable sensor assembly 700. In other examples, the stretchable shape may have sharp corners bent similar to an accordion fold rather than smooth bends of a wave shape. In other examples, the stretchable shape may be a twist or spiral, similar to a spring shape. In any examples, the stretchable shape may allow the distance between the emitters and detectors to be varied according to the size of a target, such that the stretchable sensor assembly may fit a range of target sizes with the emitters and detectors aligned for accurate measurements.

In the example where the stretchable shape is a wave shape, the resting shape taken in a relaxed state of the elastic cover may be a wave, and the maximally stretched state may be a flat ribbon. For example, second flex portion 508 of pulse oximetry hardware 500 may be deformed from a flat ribbon shape to a wave shape prior to being over molded with elastic cover 702. As a result, when the elastic material is molded onto the wave shaped second flex portion 508, elastic cover 702 takes a wave shape in third portion 724. A mold used to over mold pulse oximetry hardware 500 may also include an approximately same wave shape to form fifth portion 722 as is used to form third portion 724. Thus, elastic cover 702 takes the wave shape in third portion 724 and fifth portion 722 as a resting shape which elastic cover 702 takes in an absence of tension or compression. Thus, elastic cover 702 holds second flex portion 508 (shown in FIG. 5) in a wave shape in an absence of tension. Manipulation of elastic cover 702 by a user to fit stretchable sensor assembly 700 to a target, such as a finger, may change the shape of elastic cover 702, thereby changing a shape of pulse oximetry hardware 500, more specifically second flex portion 508. On its own, pulse oximetry hardware 500 may not be elastic such that pulse oximetry hardware returns to the compact state after being stretched. Thus, the ability to stretch stretchable sensor assembly 700 may be provided by material characteristics and the resting shape of elastic cover 702, while flex portions of pulse oximetry hardware 500 may be adjusted according to behavior of elastic cover 702.

In this way, stretchable sensor assembly 700 may fit a range of target sizes, for example finger sizes, such that stretchable sensor assembly is in face-sharing contact with the target in at least two portions (e.g., second portion 714 and fourth portion 716) and the emitters and detectors are aligned. A first ring formed by stretchable sensor assembly 700 in the relaxed state (e.g., without tension), or compact state, may serve as a lower threshold for the target size. For example, the target diameter may not be significantly smaller than a first inner diameter of the first ring. A second ring formed by stretchable sensor assembly 700 in a stretched state in which the waved portions are flattened may serve as an upper threshold for the target size. For example, the target diameter may not be greater than a second inner diameter of the second ring. Thus, the target diameter may be between and including the lower threshold and upper threshold, for example. In other examples, a range of target sizes may be determined by other measurements than diameters, such as circumferences or perimeters.

Turning to FIGS. 10 and 11, stretchable sensor assembly 700 is shown positioned on (e.g., worn on) a target 1002 in a top view 1000 and a front view 1100, respectively. When positioned on the target 1002, the stretchable sensor assembly 700 may extend about, such as around, and be in surface sharing contact with the target 1002. Therefore, stretchable sensor assembly may be a wearable system. In an example in which emitters 514 and detectors 516 (as shown in FIG. 5) are light emitters and photodetectors, respectively, stretchable sensor assembly 700 may be considered a blood oxygen sensor. Thus, stretchable sensor assembly 700 may also be a wearable blood oxygen sensor, in at least some examples. In such examples, the target 1002 may be a finger of a patient. Reference axes 1004 are provided in FIGS. 10 and 11, including an x-axis, a y-axis, and a z-axis. For example, the z-axis may be parallel to the path of light emitted from the emitters of stretchable sensor assembly 700. Additionally or alternatively, the x-axis may be parallel to an axial length of the target 1002, and the y-axis and z-axis may be parallel to radial directions of the target 1002.

As described above and shown in FIGS. 10-11, sixth portion 708 may be connected to second portion 714 when positioned on a target, such as the target 1002. Sixth portion 708 is shown connected on top (e.g., in a positive z-direction) of second portion 714 via the connecting member 1006 which may be a complementary counterpart to the hole 712 in sixth portion 708. However, in other examples, the sixth portion may connect to the second portion by other means and in other orientations. For example, a side of sixth portion 708 may be connected to a side of second portion 714 rather than the top. In at least some examples, the complementary counterpart may removably lock sixth portion 708 and second portion 714 together. In other examples, sixth portion 708 and second portion 714 may be permanently linked together, such that the ring shape may not be unfolded such as shown in FIGS. 8 and 9. Thus, a variety of connection mechanisms may be employed in order to position a stretchable sensor assembly around a target without departing from the scope of this disclosure.

Due to the connection between sixth portion 708 and second portion 714, emitters (e.g., emitters 514 of FIGS. 5-9) and detectors (e.g., detectors 516 of FIGS. 5-9) positioned within second portion 714 and fourth portion 716, respectively, may be aligned such that an accurate oxygen saturation level may be determined. As described above in reference to FIGS. 8 and 9, third portion 724 and fifth portion 722 may have approximately the same wave shape. Therefore, third portion 724 and fifth portion 722 may have approximately the same resistance to deformation, thus allowing for approximately equal distances between second portion 714 and fourth portion 716, and between fourth portion 716 and sixth portion 708 in any state (e.g., compact state 800 in FIG. 8, stretched state 900 in FIG. 9, positioned on the target 1002). Therefore, distances between the emitters and detectors around the target 1002 in either direction (e.g., clockwise and counterclockwise in FIG. 11) may be approximately the same as each other, regardless of the size (e.g., diameter) of the target 1002. Consequently, the emitters and detectors may be aligned such that light emitted from the emitters may follow a path shown by an arrow 1102 through the target and be intercepted by the detectors.

The technical effect of the stretchable sensor assembly disclosed herein is to ensure alignment of sensor components, such as emitters and detectors, when positioned around a target, for a range of sizes of the target. Additionally, the stretchable sensor assembly disclosed herein avoids having separate electric couplings for the detectors and emitters which prevents tangling and/or snagging of extra wires/cords and may reduce resource demand.

The disclosure also provides support for a device, comprising: an emitter, a detector, a power/signal cable directly connected to one of the detector or the emitter, and a wave-shaped flexible printed circuit board (PCB) disposed between the emitter and the detector, wherein the wave-shaped flexible PCB includes a first plurality of conductive traces. In a first example of the system, the emitter is a light emitting diode and the detector is a photodetector. In a second example of the system, optionally including the first example, a second flexible PCB including a second plurality of conductive traces is disposed between the power/signal cable and the emitter. In a third example of the system, optionally including one or both of the first and second examples, the wave-shaped flexible PCB is perpendicular to the second flexible PCB. In a fourth example of the system, optionally including one or more or each of the first through third examples, the device is over molded with an elastic cover that holds the wave-shaped flexible PCB in a wave shape in an absence of tension and allows the wave-shaped flexible PCB to be stretched under tension. In a fifth example of the system, optionally including one or more or each of the first through fourth examples the elastic cover further comprising a wave-shaped band extending from the detector and configured to lock onto a portion comprising the emitter. In a sixth example of the system, optionally including one or more or each of the first through fifth examples, the wave-shaped band has approximately the same wave shape as the wave-shaped flexible PCB. In a seventh example of the system, optionally including one or more or each of the first through sixth examples, the emitter and detector are electrically coupled via the conductive traces therebetween, such that the detector is electrically coupled to the power/signal cable via the emitter.

The disclosure also provides support for a wearable system, comprising: a sensor comprised of an emitter coupled to a detector via a wave-shaped flexible printed circuit board (PCB), the wave-shaped flexible PCB including conductive traces, an elastic material molded over the wave-shaped flexible PCB adapted to maintain a first distance between the emitter and detector when not under tension. In a first example of the system, the elastic material couples to itself to form a ring shape. In a second example of the system, optionally including the first example, tension increases the first distance to a second distance. In a third example of the system, optionally including one or both of the first and second examples, the emitter and the detector are electrically coupled to each other by the conductive traces, where there are no other electric couplers than the conductive traces therebetween. In a fourth example of the system, optionally including one or more or each of the first through third examples, the emitter and detector are positioned equidistantly along the ring shape. In a fifth example of the system, optionally including one or more or each of the first through fourth examples, the emitter is a light emitting diode and the detector is a photodetector. In a sixth example of the system, optionally including one or more or each of the first through fifth examples, the emitter is mounted on a first rigid PCB and the detector is mounted on a second rigid PCB, the wave-shaped flexible PCB is disposed between the first rigid PCB and the second rigid PCB.

The disclosure also provides support for a wearable blood oxygen sensor, comprising: a light emitter coupled to a first rigid printed circuit board (PCB), a photodetector coupled to a second rigid PCB, a flexible PCB, comprising conductive traces coupled at a first end to the light emitter and at a second end to the photodetector, the flexible PCB adapted to change shape to adjust a distance between the light emitter and the photodetector. In a first example of the system, the wearable blood oxygen sensor is over molded with an elastic cover that changes the shape of the flexible PCB under tension to adjust the distance between the light emitter and the photodetector. In a second example of the system, optionally including the first example, the flexible PCB is wave shaped when not under tension. In a third example of the system, optionally including one or both of the first and second examples, the wearable blood oxygen sensor is wearable on a finger. In a fourth example of the system, optionally including one or more or each of the first through third examples, the wearable blood oxygen sensor fits around a range of finger sizes such that the light emitter and the photodetector are aligned.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

FIGS. 1-12 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example. FIGS. 5-11 are shown approximately to scale, although other relative dimensions may be used.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A device, comprising:
an emitter;
a detector;
a cable directly connected to one of the detector or the emitter; and
a wave-shaped flexible printed circuit board (PCB) disposed between the emitter and the detector, wherein the wave-shaped flexible PCB includes a first plurality of conductive traces.

2. The device of claim 1, wherein the emitter is a light emitting diode and the detector is a photodetector.

3. The device of claim 1, wherein a second flexible PCB including a second plurality of conductive traces is disposed between the cable and the emitter.

4. The device of claim 3, wherein the wave-shaped flexible PCB is perpendicular to the second flexible PCB.

5. The device of claim 1, wherein the device is over molded with an elastic cover that holds the wave-shaped flexible PCB in a wave shape in an absence of tension and allows the wave-shaped flexible PCB to be stretched under tension.

6. The device of claim 5, the elastic cover further comprising a wave-shaped band extending from the detector and configured to lock onto a portion comprising the emitter, or extending from the emitter and configured to lock onto a portion comprising the detector.

7. The device of claim 6, wherein the wave-shaped band has approximately the same wave shape as the wave-shaped flexible PCB.

8. The device of claim 1, wherein the emitter and detector are electrically coupled via the first plurality of conductive traces therebetween, such that the detector is electrically coupled to the cable via the emitter.

9. A wearable system, comprising:
a sensor comprised of an emitter coupled to a detector via a wave-shaped flexible printed circuit board (PCB), the wave-shaped flexible PCB including conductive traces; and
an elastic material molded over the wave-shaped flexible PCB adapted to maintain a first distance between the emitter and detector when not under tension.

10. The wearable system of claim 9, wherein the elastic material couples to itself to form a ring shape.

11. The wearable system of claim 9, wherein tension increases the first distance to a second distance.

12. The wearable system of claim 9, wherein the emitter and the detector are electrically coupled to each other by the conductive traces, where there are no other electric couplers than the conductive traces therebetween.

13. The wearable system of claim 10, wherein the emitter and detector are positioned equidistantly along the ring shape.

14. The wearable system of claim 9, wherein the emitter is a light emitting diode and the detector is a photodetector.

15. The wearable system of claim 9, wherein the emitter is mounted on a first rigid PCB and the detector is mounted on a second rigid PCB, the wave-shaped flexible PCB is disposed between the first rigid PCB and the second rigid PCB.

16. A wearable blood oxygen sensor, comprising:
a light emitter coupled to a first rigid printed circuit board (PCB);
a photodetector coupled to a second rigid PCB; and
a flexible PCB, comprising conductive traces coupled at a first end to the light emitter and at a second end to the photodetector, the flexible PCB adapted to change shape to adjust a distance between the light emitter and the photodetector.

17. The wearable blood oxygen sensor of claim 16, wherein the wearable blood oxygen sensor is over molded with an elastic cover that changes the shape of the flexible PCB under tension to adjust the distance between the light emitter and the photodetector.

18. The wearable blood oxygen sensor of claim 16, wherein the flexible PCB is wave shaped when not under tension.

19. The wearable blood oxygen sensor of claim 16, wherein the wearable blood oxygen sensor is wearable on a finger.

20. The wearable blood oxygen sensor of claim 19, wherein the wearable blood oxygen sensor fits around a range of finger sizes such that the light emitter and the photodetector are aligned.
